(19) United States
(12) Patent Application Publication
(10) Pub. No.: US 2026/0134538 A1
(43) Pub. Date: May 14, 2026

(54) SLIDE SCANNING DEVICE HAVING DOOR CONFIGURED TO INCLUDE DISPLAY

(71) Applicant: CURIOSIS CO., LTD., Seoul (KR)

(72) Inventors: Minseop PARK, Seoul (KR); Jae Hoon SHIN, Seoul (KR); Beom Jun LEE, Seoul (KR)

(73) Assignee: CURIOSIS CO., LTD., Seoul (KR)

(21) Appl. No.: 19/433,337

(22) Filed: Dec. 26, 2025

Related U.S. Application Data
(63) Continuation of application No. PCT/KR2024/008589, filed on Jun. 21, 2024.

(30) Foreign Application Priority Data
Jun. 26, 2023 (KR) 10-2023-0081691
Jul. 25, 2023 (KR) 10-2023-0096775
Dec. 26, 2023 (KR) 10-2023-0191330

(51) Int. Cl. G06T 7/00; G01N 35/00

(52) U.S. Cl. CPC G06T 7/0012; G01N 35/00029; G01N 35/00722; G01N 2035/00138; G01N 2035/0091; G06T 2207/10056; G06T 2207/30024

(57) ABSTRACT

The purpose of an embodiment of the present disclosure is to provide a slide scanning device comprising: a memory; at least one processor which executes one or more instructions stored in the memory; and a door which includes a display, wherein the at least one processor identifies a door state including at least one of a closed state of the door, an opened state of the door, and a fully opened state of the door, and on the basis of the door state, controls the display to output a user interface (UI) corresponding to the door state.

### CROSS-REFERENCE TO RELATED APPLICATIONS

This application is a continuation of International Application No. PCT/KR2024/008589 filed on Jun. 21, 2024, which claims priority to Korean Patent Application No. 10-2023-0081691 filed on Jun. 26, 2023, Korean Patent Application No. 10-2023-0096775 filed on Jul. 25, 2023 and Korean Patent Application No. 10-2023-0191330 filed on Dec. 26, 2023, the entire contents of which are herein incorporated by reference.

### TECHNICAL FIELD

The present disclosure relates to a slide scanning apparatus, and more particularly, to a slide scanning apparatus in which a door is configured to include a display.

### BACKGROUND ART

Pathology departments play a role in providing the foundation for clinical treatment decisions by observing and analyzing morphological changes in a patient's cells, tissues, and bodily fluids to make diagnoses. Conventionally, in pathology departments, cytological or histological examinations have been performed by a specialist who visually interprets and diagnoses a patient's cell slide or tissue slide using a microscope. Recently, however, as digital slide scanners have been introduced into pathological diagnosis, pathological diagnosis has been performed not only through a microscope but also through digital images obtained by scanning a stained cell slide or stained tissue slide.

Slides containing a patient's specimen are accommodated in a slide tray, and the slide tray is accommodated in a slide tray slot. A digital slide scanner or the like scans the slide tray accommodated in the slide tray slot, and as the scanning is performed, information regarding the slides accommodated in the slide tray is obtained, enabling pathological diagnosis to be carried out.

Accordingly, information regarding a plurality of slides can be obtained within a short period of time, and it is also important to provide the scan information in a manner that allows a user to conveniently identify the information. Therefore, existing slide scanning apparatuses provide the scan information of slides by connecting a separate monitor to the outside of the slide scanning apparatus or locate a display screen portion and a slide insertion portion at separate positions. However, when a separate monitor is provided or when the display screen portion and the slide insertion portion are separately located, it becomes difficult to reduce the size of the slide scanning apparatus, resulting in inconvenience in moving or installing the scanning apparatus. Therefore, a slide scanning apparatus capable of being miniaturized is required.

### SUMMARY

### Technical Objective

The objective of the present disclosure is to provide a slide scanning apparatus in which a door is configured to include a display.

### Means for Solving Technical Objective

The present disclosure aims to provide a slide scanning apparatus in which a door is configured to include a display such as a monitor.

A slide scanning apparatus according to an embodiment of the present disclosure to achieve the above technical objective comprises a memory, at least one processor configured to execute at least one instruction stored in the memory, and a door comprising a display. In an embodiment, the at least one processor is configured to identify a door state comprising at least one of a closed state of the door, an open state of the door, or a fully-open state of the door, and to control the display to output a user interface (UI) corresponding to the door state based on the door state.

In an embodiment, the door may be slidably installed.

In an embodiment, the slide scanning apparatus comprises a slide tray slot, and the slide tray may be extracted from or inserted into the slide tray slot when the door is in the fully-open state.

In an embodiment, the slide scanning apparatus further comprises a first door damper, a second door damper, a first door lock, and a second door lock, and the door may be fixed by the first door damper and the first door lock when the door is in the closed state, and the door may be fixed by the second door damper and the second door lock when the door is in the fully-open state.

In an embodiment, the display includes a touch panel, and the at least one processor may be configured not to acquire a user's touch input to a specific region of the display when the door is in the open state or the fully-open state.

In an embodiment, the slide scanning apparatus comprises a slide tray slot, and the user interface corresponding to the door state comprises a first GUI regarding one or more slide trays located in the slide tray slot and a second GUI regarding one or more slides located on the slide tray. The first GUI and the second GUI comprise one or more GUIs configured to acquire a touch input when the door is in the closed state, and provide the corresponding GUI not to acquire a touch input when the door is in the open state or the fully-open state.

In an embodiment, the door further comprises a bracket configured to support the display.

In an embodiment, the at least one processor may be configured to control the display to provide a UI for opening the door or a UI for closing the door.

In an embodiment, the slide scanning apparatus further comprises a door lock device, and the at least one processor may be configured to control the door lock device to unlock the door lock device when obtaining a user input to a UI for opening the door or a UI for closing the door.

In an embodiment, the slide scanning apparatus further comprises at least one of a forced lock-release hole or a forced fixation-release hole.

In an embodiment, the slide scanning apparatus further comprises a locking device including a solenoid switch.

In an embodiment, the door may be unlocked when an external force is applied through the forced lock-release hole such that a first operation bar rotates in a first direction.

In an embodiment, the door may be unlocked when an external force is applied through the forced fixation-release hole such that a second operation bar rotates in a second direction.

In an embodiment, the first GUI is formed to correspond to an arrangement of a plurality of slide tray accommodation units in the slide tray slot, and the first GUI comprises a GUI indicating whether a slide tray is inserted into each of the plurality of slide tray accommodation units. In the first GUI, a GUI regarding the slide tray displayed through the second GUI may be formed larger than GUIs regarding the other slide trays.

In an embodiment, the first GUI comprises a GUI indicating at least one of location information of the one or more slide trays inserted into the slide tray slot, slide tray scan progress information regarding at least a portion of the one or more slide trays, slide tray absence information, slot identification information, slide tray accommodation unit identification information, or slide tray identification information of the one or more slide trays. The second GUI comprises a GUI indicating at least one of location information of the one or more slides on the slide tray, slide scan progress information regarding at least a portion of the one or more slides, specimen information of the one or more slides, code information of the one or more slides, slide tray identification information, or slide identification information of the one or more slides.

In an embodiment, the slide scanning apparatus further comprises a preview camera configured to obtain a preview image of a slide tray, a scan camera configured to obtain a slide scan image, and a slide tray stage configured to transport a slide tray.

In an embodiment, the at least one processor may obtain a preview image of a slide tray, recognize information regarding the slide tray, slides, or pathological samples from the preview image, obtain the slide scan image based on the information regarding the slide tray, the slides, or the pathological samples, and store the slide scan image in association with the information regarding the slide tray, the slides, or the pathological samples.

In an embodiment, the information regarding the slide tray may comprise whether the slide tray is present on the slide tray stage, identification information of the slide tray, orientation information of the slide tray, or identification information of each accommodation portion of the slide tray.

In an embodiment, the information regarding the slide may comprise whether a slide is present in each slide accommodation portion, whether a slide in each slide accommodation portion is damaged, whether a slide is properly accommodated in each slide accommodation portion, or information regarding a region of interest in which a pathological sample is present in the slide.

In an embodiment, the information regarding the pathological sample may comprise a specimen site of the pathological sample, a specimen collection time, whether staining has been performed, a name of a staining reagent, or patient information corresponding to the pathological sample.

In an embodiment, the patient information corresponding to the pathological sample may comprise a patient name, a patient age, a patient's national identification number, a patient number, a medical department of the patient, or a disease name of the patient.

In an embodiment, the information regarding the pathological sample may be obtained by recognizing characters, numbers, symbols, a barcode, or a QR code recorded in an information inscription portion of each slide.

In an embodiment, information regarding the slide tray, the slides, or the pathological samples may be provided to a user together with the preview image, an input of a selection regarding slide scanning may be received from the user, and a slide scan image may be obtained based on the user input.

In an embodiment, the information regarding the pathological sample may comprise information regarding a priority of the pathological sample, a scan resolution, a scan area, or a scan method.

In an embodiment, the slide tray stage may include a marker located at a predetermined position, and the marker may be obscured when the slide tray is accommodated on the slide tray stage.

In an embodiment, the slide scan image may be transmitted to an external device based on the information regarding the slide tray, the slides, or the pathological samples.

In an embodiment, a slide scanning method comprises: obtaining a preview image of a slide tray; recognizing information regarding the slide tray, slides, or pathological samples from the preview image; obtaining a slide scan image based on the information regarding the slide tray, the slides, or the pathological samples; and storing the slide scan image in association with the information regarding the slide tray, the slides, or the pathological samples.

In an embodiment, the information regarding the slide tray may comprise whether the slide tray is present on a slide tray stage, identification information of the slide tray, orientation information of the slide tray, or identification information of each accommodation portion of the slide tray.

In an embodiment, the information regarding the slide may comprise whether a slide is present in each slide accommodation portion, whether the slide in each slide accommodation portion is damaged, whether the slide is properly positioned in each slide accommodation portion, or information regarding a region of interest in which a pathological sample is present within the slide.

In an embodiment, the information regarding the pathological sample may comprise a specimen site of the pathological sample, a specimen collection time, whether staining has been performed, a name of a staining reagent, or patient information corresponding to the pathological sample.

In an embodiment, the patient information corresponding to the pathological sample may comprise a patient name, a patient age, a patient's national identification number, a patient number, a medical department of the patient, or a disease name of the patient.

In an embodiment, the information regarding the pathological sample may be obtained by recognizing characters, numbers, symbols, a barcode, or a QR code recorded in an information inscription portion of each slide.

In an embodiment, the slide scanning method may further comprise: providing the information regarding the slide tray, the slide, or the pathological sample together with the preview image to a user; and receiving an input from the user regarding a selection for slide scanning, wherein the step of obtaining the slide scan image based on the information about the slide tray, the slide, or the pathological sample may be performed based on the user input.

In an embodiment, the information regarding the pathological sample may comprise information regarding a priority of the pathological sample, a scan resolution, a scan area, or a scan method.

In an embodiment, the slide tray stage may include a marker located at a predetermined position, and the marker may be obscured when the slide tray is accommodated on the slide tray stage.

In an embodiment, the slide scanning method may further include the step of transmitting the slide scan image to an external device based on the information regarding the slide tray, the slides, or the pathological samples.

In an embodiment, the present disclosure includes a program stored on a recording medium to cause a computer to execute a method according to one embodiment of the present disclosure.

In an embodiment, the present disclosure includes a computer-readable recording medium having recorded thereon a program for executing a method according to one embodiment of the present disclosure on a computer.

In an embodiment, the present disclosure includes a computer-readable recording medium having recorded thereon a database used in one embodiment of the present disclosure.

### Advantageous Effect

According to an embodiment of the present disclosure, a slide scanning apparatus in which a monitor (i.e., a display screen unit) is integrally mounted on a door of the slide scanning apparatus can be provided, thereby enabling miniaturization of the slide scanning apparatus.

According to an embodiment of the present disclosure, even in an emergency such as a power failure, a slide tray accommodated in the slide scanning apparatus may be extracted or inserted with equipment capable of opening or closing the door of the slide scanning apparatus in an emergency. According to an embodiment of the present disclosure, operational stability of the slide scanning apparatus can be improved by controlling the apparatus such that a touch input is not received when the door is in the open state.

According to an embodiment of the present disclosure, image scanning of a pathological sample can be optimized by collecting information regarding the slide(s) before image scanning of the pathological sample.

According to an embodiment of the present disclosure, information regarding a pathological sample can be efficiently collected from the slide(s).

According to an embodiment of the present disclosure, a state of the slide and/or the slide scanning apparatus can be checked using a preview image of the slide tray.

In addition to the foregoing, specific effects of the present disclosure will be described together while explaining detailed matters for carrying out the disclosure below.

### BRIEF DESCRIPTION OF DRAWINGS

FIG. 1A and FIG. 1B are views illustrating a slide scanning apparatus according to an embodiment of the present disclosure.

FIG. 2A and FIG. 2B are exploded views of a door of a slide scanning apparatus in a closed state according to an embodiment of the present disclosure.

FIG. 3A and FIG. 3B are views illustrating a method for forcibly releasing a lock of the door in an emergency according to an embodiment of the present disclosure.

FIG. 4 is an exploded view of a door of a slide scanning apparatus in a fully-open state according to an embodiment of the present disclosure.

FIG. 5A and FIG. 5B are views illustrating a method for forcibly releasing fixation of the door in an emergency according to an embodiment of the present disclosure.

FIG. 6 is a view illustrating a UI/UX in a closed state of the door according to an embodiment of the present disclosure.

FIG. 7 is a view illustrating a UI/UX in an open state of the door according to an embodiment of the present disclosure.

FIG. 8 is a view illustrating a UI/UX in a fully-open state of the door according to an embodiment of the present disclosure.

FIG. 9 is a block diagram of a slide scanning apparatus according to an embodiment of the present disclosure.

FIG. 10 is a view illustrating a slide scanning apparatus according to an embodiment of the present disclosure.

FIG. 11 is a view illustrating a slide tray according to an embodiment of the present disclosure.

FIG. 12 is a view illustrating a slide according to an embodiment of the present disclosure.

FIG. 13 is a view illustrating a slide scanning apparatus according to an embodiment of the present disclosure.

FIG. 14 is a view illustrating a slide scanning method according to an embodiment of the present disclosure.

### DETAILED DESCRIPTION OF THE INVENTION

To clarify the technical spirit of the present disclosure, embodiments of the present disclosure will be described in detail with reference to the accompanying drawings. In describing the present disclosure, detailed descriptions of known functions or components that may unnecessarily obscure the gist of the present disclosure will be omitted. Although components having substantially the same functional configuration are shown in different drawings, the same reference numerals are assigned to the components wherever possible. For convenience of explanation, a device and a method may be described together where appropriate. The operations of the present disclosure need not necessarily be performed in the order described and may be performed in parallel, selectively, or individually.

Terms used in embodiments of the present disclosure have been selected from commonly used terms in consideration of the functions of the present disclosure; however, such terms may vary depending on the intent of a person skilled in the art, precedents, or the emergence of new technologies. In certain cases, terms arbitrarily selected by the applicant may be used, and in such cases, the meanings thereof will be described in detail in the description of the corresponding embodiments. Therefore, the terms used in the present specification should be interpreted based on the meanings thereof and the overall content of the present disclosure rather than merely on their names.

Throughout the present disclosure, singular expressions may include plural expressions unless the context clearly indicates otherwise. The terms “comprise” and “have” indicate the presence of a feature, a number, a step, an operation, a component, a part, or a combination thereof, but do not preclude the possibility of the presence or addition of one or more other features, numbers, steps, operations, components, parts, or combinations thereof. That is, when a component “comprises” another component in the present disclosure, the statement does not exclude the possibility that additional components may be included unless otherwise stated.

Expressions such as “at least one of” modify an entire list of elements and do not modify the individual elements separately. For example, “at least one of A, B, and C” or “at least one of A, B, or C” refers to only A, only B, only C, both A and B, both B and C, both A and C, all of A, B, and C, or a combination thereof.

The terms “. . . unit” and “. . . module” used in the present disclosure refer to units that perform at least one function or operation, and may be implemented in hardware, software, or a combination of hardware and software.

Throughout the present disclosure, when a part is described as being “connected” to another part, it should be understood to include not only a direct connection but also an electrical connection through another component interposed therebetween. When a part “comprises” a component, this means that other components may be further included unless otherwise stated.

The expression “configured to” (or “set to”) used throughout the present disclosure may be replaced, depending on context, with expressions such as “suitable for,” “having the capacity to,” “designed to,” “adapted to,” “made to,” or “capable of.” The term “configured to” does not necessarily mean “specifically designed to” in a hardware-exclusive sense. In certain situations, “a processor configured to perform A, B, and C” may refer to a dedicated processor (e.g., an embedded processor) or a general-purpose processor (e.g., a CPU or application processor) that performs the operations by executing one or more software programs stored in memory.

Throughout the present disclosure, a slide refers to a thin and flat piece of glass used to fix a specimen, and may include, but is not limited to, a slide glass, a glass slide, a specimen slide, a microscope slide, or a slide glass on which a specimen is mounted and covered with a cover glass.

Throughout the present disclosure, a slide tray refers to a case for carrying or storing slides, and may also be referred to as a tray, a glass plate, a slide plate, a slide holder, a glass holder, a slide case, a glass case, a slide folder, or a glass folder.

Throughout the present disclosure, a slide tray slot refers to a device for storing slide trays, and may also be referred to as a slide tray cabinet, a slide tray storage cabinet, or a slide tray rack.

The term “specimen” used in the present disclosure may be used interchangeably with “sample” and includes all materials known in the art to be observable through an optical or light microscope. For example, the specimen may include, but is not to limited to, cells or tissues (e.g., cells or tissues of animals, plants, fungi, protists, bacteria, etc.—including both normal or pathological cells/tissues), cell components (e.g., nuclei, cytoplasm, chloroplasts, mitochondria, etc.), microorganisms (e.g., bacteria, protozoa, certain algae, fungi, etc.), and organoids.

The term “about,” as used in the present disclosure, refers to within 10%, preferably within 5%, and more preferably within 1% of a given numerical value or range.

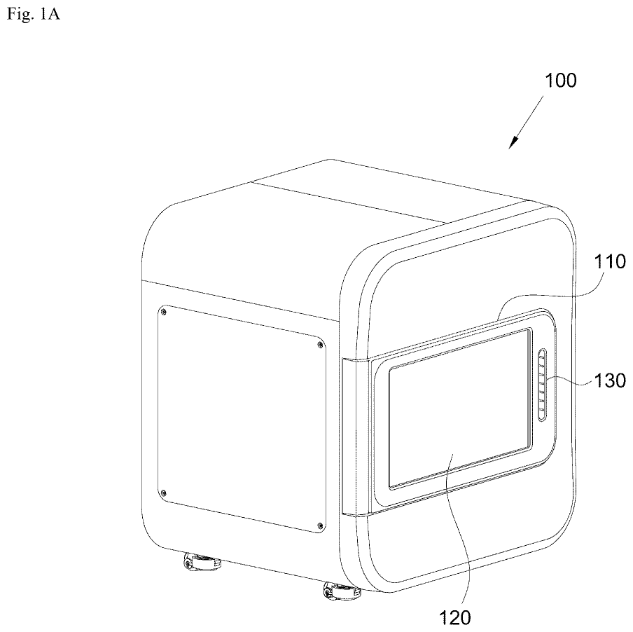

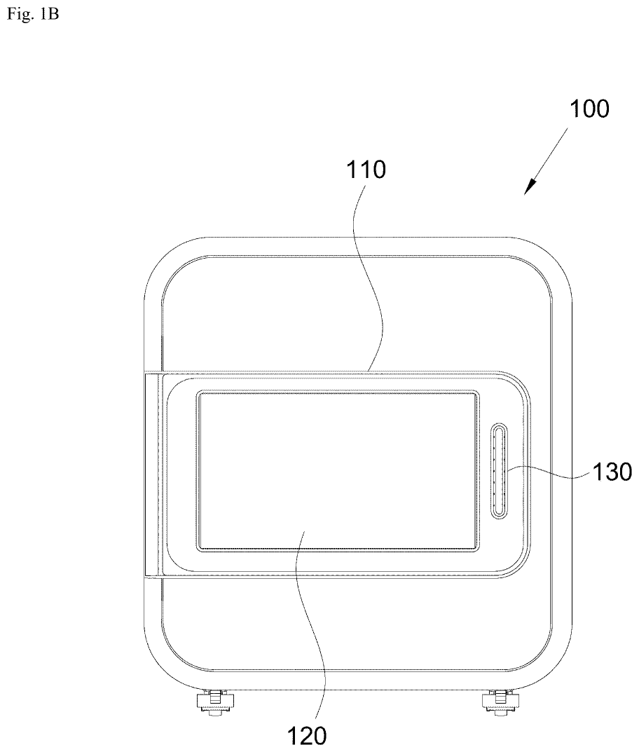

FIG. 1A and FIG. 1B are views illustrating a slide scanning apparatus according to an embodiment of the present disclosure.

Referring to FIG. 1A and FIG. 1B, a slide scanning apparatus 100 may comprise a door 110 that is slidably installed. In an embodiment, the door 110 may comprise a display 120 and a handle 130. For example, the display 120 may comprise a touch monitor or a touch screen. In addition, the display 120 may be fixed to a bracket and function as the door.

In an embodiment, it may be designed such that information displayed on the display 120 varies depending on whether the door 110 is in the fully-open state, the open state, or the closed state. That is, the slide scanning apparatus 100 may identify at least one of a closed state of the door, an open state of the door, or a fully-open state of the door, and control the display 120 to output a user interface (UI) corresponding to the door state based on the door state. For example, when the door 110 is in the open state or the fully-open state, the slide trays may be exposed externally or their safety may not be ensured due to movements of equipment. Thus, it may be controlled such that scanning operations of the slide scanning apparatus 100 are disabled. Accordingly, when the door 110 is in the open state or the fully-open state, the display 120 may output information indicating that scanning of slides is not possible due to the door being in the open state.

According to an embodiment, unlike conventional apparatuses that require a large installation space by providing slide scan information on a separate monitor, the slide scanning apparatus may be miniaturized by having the monitor integrated into the form of a door.

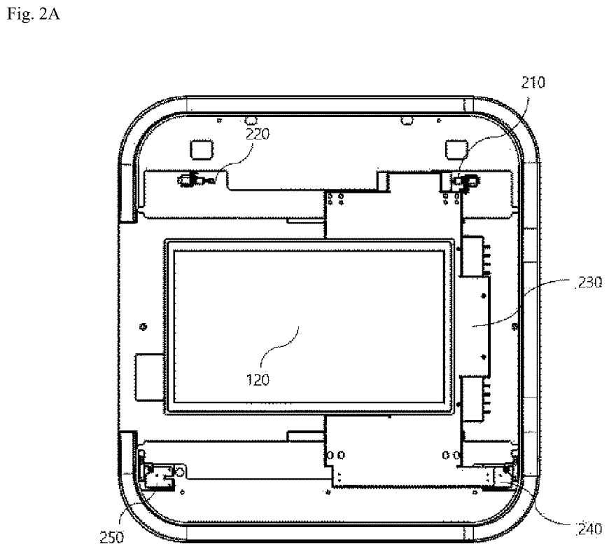

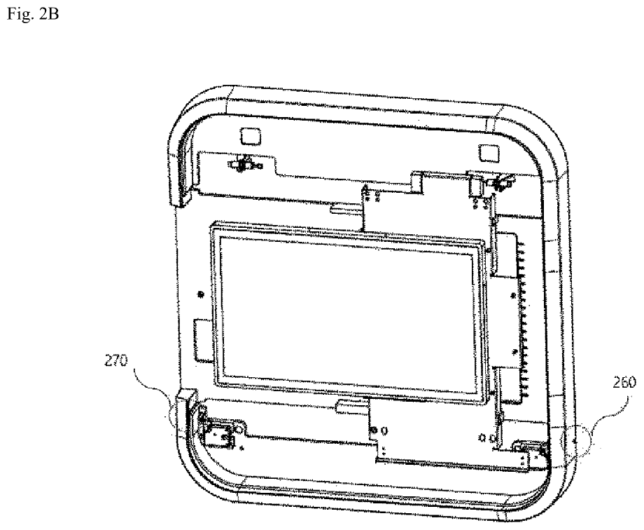

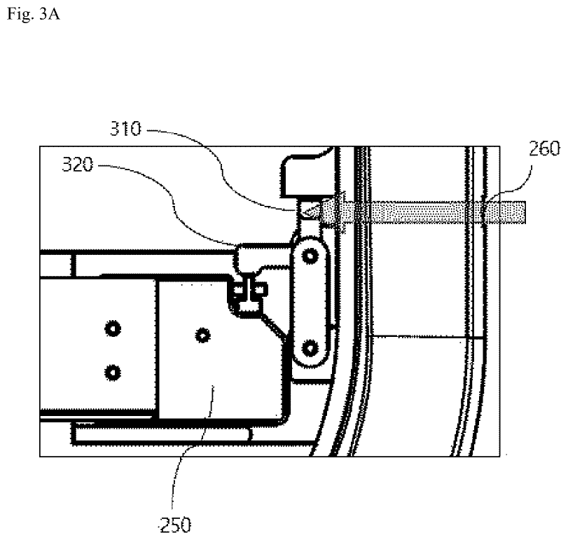

FIG. 2A and FIG. 2B are exploded views of a door of a slide scanning apparatus in a closed state according to an embodiment of the present disclosure.

Referring to FIG. 2A and FIG. 2B, the slide scanning apparatus 100 may comprise a first door damper 210, a second door damper 220, a display 120, a bracket 230 configured to support the display, a first door lock 240, a second door lock 250, a forced lock-release hole 260, a forced fixation-release hole 270, etc. However, not all components illustrated in FIG. 2A are essential components of the slide scanning apparatus 100. The slide scanning apparatus 100 may be implemented with more components than those shown in FIG. 2A, or may be implemented with fewer components than those shown in FIG. 2A.

In an embodiment, when the bracket 230 engages with the first door damper 210, the door is defined as being in the closed state, and when the bracket 230 engages with the second door damper 220, the door is defined as being in the fully-open state. When the bracket 230 is located in a section between the closed state and the fully-open state, the door is defined as being in the open state. Accordingly, the open state of the door may include both a case in which the door is in a transition section from the closed state to the fully-open state and a case in which the door is in a transition section from the fully-open state to the closed state. In addition, when the door engages with the first door damper 210, the door may be fixed in the closed state by the first door lock 240, and when the door engages the second door damper 220, the door may be fixed in the fully-open state by the second door lock 250.

According to an embodiment, when the door is in the closed state, the door may be fixed in the closed state by the first door lock 240, thereby enhancing stability and safety of the slide scanning apparatus during operation. When the door is in the fully-open state, the door may be fixed by the second door lock 250, thereby improving user convenience when inserting or extracting a slide tray.

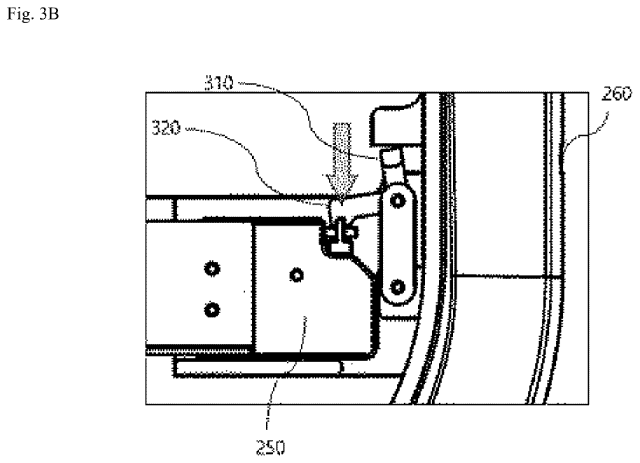

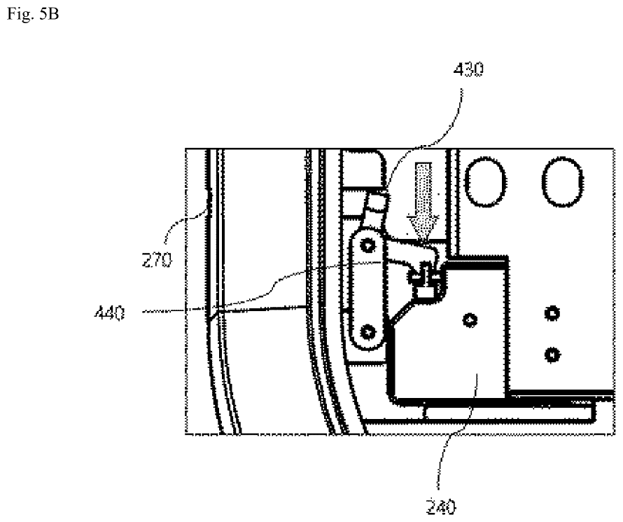

In an embodiment, the door of the slide scanning apparatus may be opened by releasing a lock in a closed state of the door, or may be closed by releasing fixation in a fully open state of the door, even in an emergency situation such as a power failure, through a forced lock-release hole (260) and a forced fixation-release hole (270). Related descriptions will be provided in further detail with reference to FIGS. 3A, FIG. 3B, FIG. 5A, and FIG. 5B.

FIG. 3A and FIG. 3B are views illustrating a method for forcibly releasing a lock of the door in an emergency according to an embodiment of the present disclosure.

Referring to FIG. 3A and FIG. 3B, the slide scanning apparatus 100 may comprise a forced lock-release hole 260. In an embodiment, when the slide scanning apparatus 100 obtains a touch input to a UI, the slide scanning apparatus 100 may release a lock of the first door lock 240 or may perform a locking procedure. For example, when the slide scanning apparatus 100 obtains a user input to a UI for opening the door or a UI for closing the door, the slide scanning apparatus 100 may control a door lock device to unlock the door lock device. However, in an emergency such as a power failure, a touch input may not be obtained, and therefore, unlocking or locking of the first door lock 240 may not be possible even when unlocking or locking is required. Accordingly, the slide scanning apparatus 100 may further comprise an emergency device configured to perform unlocking or locking of the first door lock 240 even in an emergency.

In an embodiment, a user may apply a force to rotate a first operation bar 310 in a first direction (for example, a counterclockwise direction) by inserting a rod or the like into the forced lock-release hole 260. Accordingly, when an external force applied through the forced lock-release hole causes the first operation bar 310 to rotate in the first direction, a second operation bar 320 may also rotate in the first direction, thereby unlocking the first door lock 240. For example, the emergency device may comprise a solenoid switch, and the lock of the door (i.e., a locked state) may be released when the second operation bar 320 presses the solenoid switch.

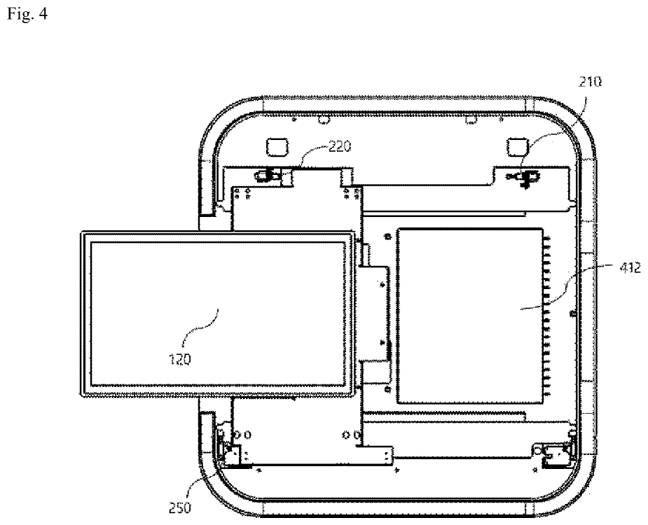

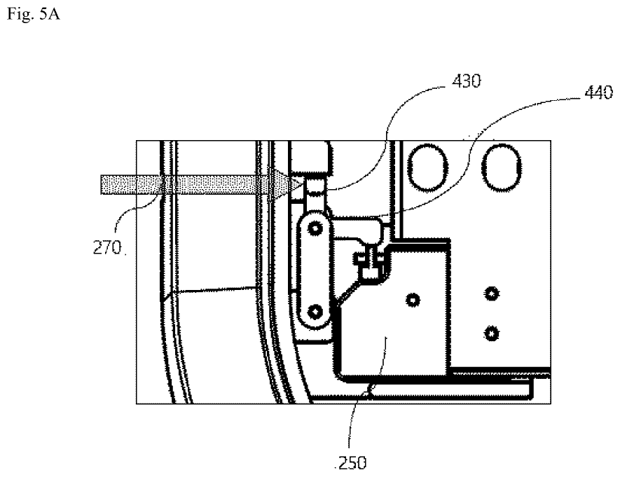

FIG. 4 is an exploded view of a door of a slide scanning apparatus in a fully-open state according to an embodiment of the present disclosure.

Referring to FIG. 4, a slide scanning apparatus 100 in a fully-open state of the door is illustrated. The fully-open state of the door may correspond to a case in which a display bracket engages with the second door damper 220. In this case, the door may be fixed in the fully-open state by the second door lock 250. In an embodiment, when the door is in the fully-open state, a user may access a slide tray accommodation space 412. A slide tray slot may be present in the slide tray accommodation space 412, and the user may insert or extract a slide tray into or from each slide tray accommodation unit.

FIG. 5A and FIG. 5B are views illustrating a method for forcibly releasing fixation of the door in an emergency according to an embodiment of the present disclosure.

Referring to FIG. 5A and FIG. 5B, the slide scanning apparatus 100 may comprise a forced fixation-release hole 270. In an embodiment, when the slide scanning apparatus 100 obtains a touch input to a UI, the slide scanning apparatus 100 may release a lock of the second door lock 250 or may perform a locking procedure. However, in an emergency such as a power failure, a touch input may not be obtained, and therefore, unlocking or locking of the second door lock 250 may not be possible even when unlocking or locking is required. Accordingly, the slide scanning apparatus 100 may further comprise an emergency device configured to perform unlocking or locking of the second door lock 250 even in an emergency.

In an embodiment, a user may apply a force to rotate a third operation bar 430 in a second direction (for example, a clockwise direction) by inserting a rod or the like into the forced fixation-release hole 270. Accordingly, when an external force applied through the forced fixation-release hole causes a third operation bar 430 to rotate in the second direction, a fourth operation bar 440 may also rotate in the second direction, thereby unlocking the second door lock 250. Unlocking of the second door lock 250 may include releasing fixation of the door in the fully-open state. For example, the emergency device may comprise a solenoid switch, and fixation of the door in the fully-open state (i.e., a locked state of the door) may be released when the fourth operation bar 440 presses the solenoid switch.

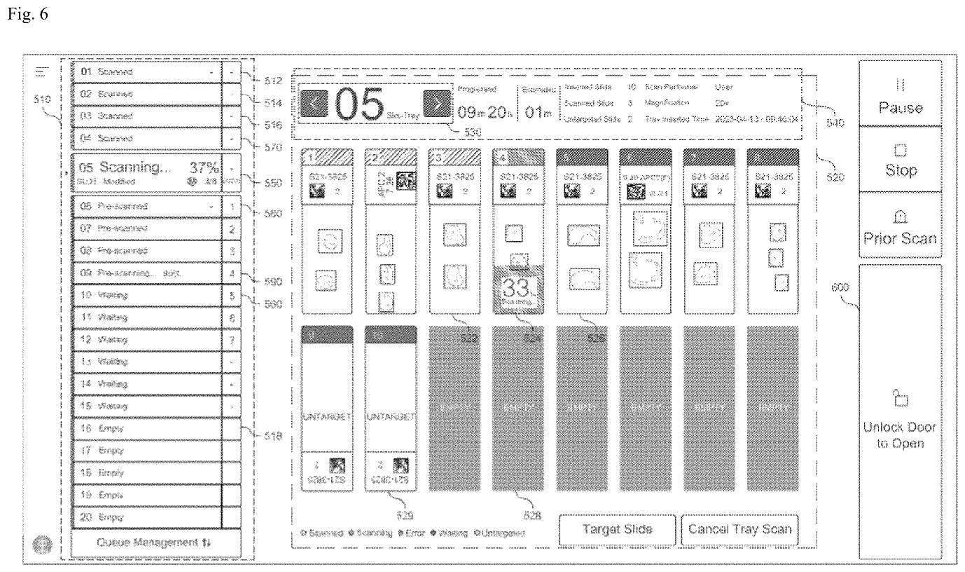

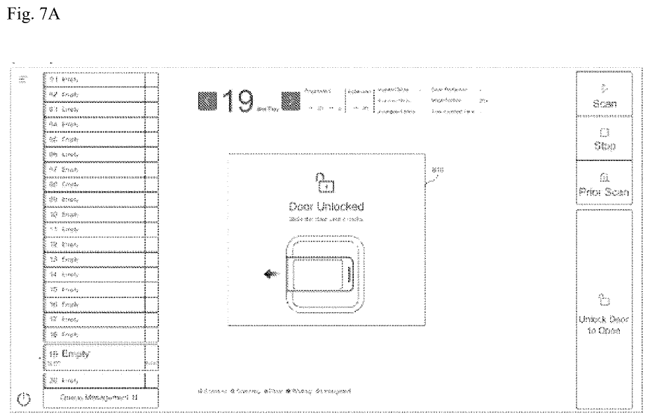

FIG. 6 is a view illustrating a UI/UX in a closed state of the door according to an embodiment of the present disclosure.

Referring to FIG. 6, the slide scanning apparatus 100 may control the display 120 to output various graphical user interfaces (GUIs) 510. In an embodiment, the display 120 may be a display device provided on the door of the slide scanning apparatus 100.

In an embodiment, GUIs that are generated by the slide scanning apparatus 100 and controlled to be output to the display 120 may comprise: a first GUI 510 regarding one or more slide trays located in a slide tray slot; a second GUI 520 regarding one or more slides located in a slide tray; and a third GUI 530 for selecting a slide tray other than the slide tray corresponding to information currently displayed through the second GUI 520.

In an embodiment, the slide scanning apparatus 100 may identify that one or more slide trays are inserted into the slide tray slot. For example, the slide tray slot may comprise an entire slot body and a plurality of slide tray accommodation units, and each slide tray may be inserted into a corresponding slide tray accommodation unit. In this case, insertion of a slide tray may be detected by a sensor provided in each of the plurality of slide tray accommodation units, and information regarding slide tray accommodation units into which the slide trays have been inserted may be received from the slide tray slot. In addition, each of the plurality of slide tray accommodation units may comprise a light emitting element, and the slide tray slot may transmit light information of the light emitting elements to the slide scanning apparatus 100. Accordingly, the slide scanning apparatus 100 may identify whether a slide tray is accommodated in each of the plurality of slide tray accommodation units, and may identify which color of light is emitted from each of the plurality of slide tray accommodation units.

In an embodiment, the slide scanning apparatus 100 may identify one or more slide trays inserted into the slide tray slot and, based on the identified one or more slide trays, may generate a first GUI 510 regarding one or more slide trays located in the slide tray slot. For example, the first GUI 510 may comprise a GUI indicating location information of the one or more slide trays inserted into the slide tray slot, slide tray scan progress information regarding at least a portion of the one or more slide trays, slide tray absence information, slot identification information, identification information of the one or more slide trays, a scan order of the slide trays, or a queue management mode.

In an embodiment, a first GUI 510 indicating location information of the one or more slide trays inserted into the slide tray slot may be formed to correspond to slide tray accommodation units arranged as a vertical stack in the slide tray slot. For example, when a first slide tray accommodation unit, a second slide tray accommodation unit, and a third slide tray accommodation unit are the three slide tray accommodation units from the top in the slide tray slot, the first GUI 510 may comprise a GUI 512 corresponding to the first slide tray accommodation unit, a GUI 514 corresponding to the second slide tray accommodation unit, and a GUI 516 corresponding to the third slide tray accommodation unit, which are sequentially formed from the top. According to an embodiment, the slide scanning apparatus 100 may provide an intuitive UI/UX by synchronizing positions of slide tray accommodation units or slide trays in the slide tray slot with GUIs.

In an embodiment, the first GUI 510 indicating slide tray scan progress information regarding at least a portion of the one or more slide trays may comprise: an indication that scanning has been completed for each slide tray (scanned); an indication that scanning is in progress (scanning); an indication that pre-scanning has been completed (pre-scanned); an indication that pre-scanning is in progress (pre-scanning); or an indication that scanning has not been started (waiting). Pre-scanning refers to a process of acquiring a preview image before scanning each slide located in a slide tray in order to recognize information regarding the slide tray, the slides, or pathological samples. For example, information regarding the slide may comprise whether a slide is present in each slide accommodation portion, whether a slide in each slide accommodation portion is damaged, whether a slide is properly accommodated in each slide accommodation portion, or information regarding a region of interest in which a pathological sample is present in the slide. Information regarding the pathological sample may comprise a specimen site of the pathological sample, a specimen collection time, whether staining has been performed, a name of a staining reagent, or patient information corresponding to the pathological sample. In addition, when scanning or pre-scanning of the slide tray is in progress, the scan progress information may comprise information regarding a progress degree of scanning or pre-scanning. For example, the first GUI 510 may comprise a GUI indicating that a fifth slide tray from the top of the slide tray slot is being scanned and that a current progress is 37%, and a GUI indicating that a ninth slide tray from the top of the slide tray slot is being pre-scanned and that a current progress is 80%.

In an embodiment, the first GUI 510 may comprise at least one of: GUIs 512, 514, and 516 corresponding to a first slide tray for which scanning has been completed; a GUI 550 corresponding to a second slide tray being scanned; GUIs 580 and 590 corresponding to a third slide tray for which pre-scanning has been completed or is in progress; a GUI 560 corresponding to a fourth slide tray for which scanning has not yet been performed; a GUI 518 indicating that no slide tray is present in a slide tray accommodation unit; and a GUI indicating a slide tray that is not a scan target. Throughout the present disclosure, a “slide tray being scanned” may refer to a slide tray for which a scanning procedure regarding at least one slide present in the slide tray is in progress. A GUI corresponding to a slide tray being scanned may comprise a GUI indicating a scanning progress degree for slides present in the slide tray. For example, the GUI 550 corresponding to the second slide tray being scanned in FIG. 6 may be configured to indicate that the scan has been completed by 37%. In another example, the GUI 590 corresponding to the third slide tray being pre-scanned in FIG. 6 may be configured to indicate that the pre-scan has been completed by 80%.

In an embodiment, the GUIs 512, 514, and 516 corresponding to the first slide tray for which scanning has been completed, the GUI 550 corresponding to the second slide tray being scanned, the GUIs 580 and 590 corresponding to the third slide tray for which pre-scanning has been completed or is in progress, the GUI 560 corresponding to the fourth slide tray for which scanning has not yet been performed, the GUI 518 indicating that no slide tray is present in a slide tray accommodation unit, and the GUI indicating a slide tray that is not a scan target may be configured to be distinguishable from one another. For example, GUIs corresponding to the first or second slide tray for which scanning has been completed or is in progress may be configured in green, GUIs 580 and 590 corresponding to the third slide tray for which pre-scanning has been completed or is in progress may be configured in yellow, and the GUI 560 corresponding to the fourth slide tray for which scanning has not yet been performed, the GUI 518 indicating that no slide tray is present in a slide tray accommodation unit, and the GUI (not shown) indicating a slide tray that is not a scan target may be configured in achromatic colors.

In an embodiment, the slide scanning apparatus 100 may obtain a user input for clicking or touching a GUI corresponding to a slide tray from the first GUI 510, which includes GUIs 512, 514, and 516 corresponding to the first slide tray for which scanning has been completed, a GUI 550 corresponding to the second slide tray being scanned, GUIs 580 and 590 corresponding to the third slide tray for which pre-scanning has been completed or is in progress, a GUI 560 corresponding to the fourth slide tray for which scanning has not yet been performed, a GUI 518 indicating that no slide tray is present in a slide tray accommodation unit, and a GUI indicating a slide tray that is not a scan target. In an embodiment, the slide scanning apparatus 100 may receive a user input for clicking or touching the GUI 550 corresponding to the second slide tray. In addition, based on the user input, the slide scanning apparatus 100 may generate the second GUI 520 corresponding to the second slide tray and control the display 120 to output the second GUI 520. Furthermore, the slide scanning apparatus 100 may update the first GUI 510 based on the user input. For example, the slide scanning apparatus 100 may update the first GUI 510 by forming the GUI 550 within the first GUI 510 corresponding to the second slide tray larger than GUIs corresponding to other slide trays.

Furthermore, the first GUI 510 may comprise a GUI 518 indicating slide tray absence information, and GUIs indicating identification information of a slot, identification information of a slide tray accommodation unit, and identification information of a slide tray. In an embodiment, the identification information of the slide tray accommodation unit may comprise an identification number of a slide tray accommodation unit and color information of light emitted from a light emitting element of the slide tray accommodation unit. According to an embodiment, the slide scanning apparatus 100 may provide an intuitive UI/UX to a user by synchronizing colors of light emitted from light emitting elements of the slide tray accommodation units with colors of UIs corresponding to the slide tray accommodation units within the first GUI 510.

In an embodiment, the slide scanning apparatus 100 may identify that one or more slide trays are inserted into the slide tray slot. For example, the slide scanning apparatus 100 may scan the slide tray slot and obtain location information of slide trays inserted into the slide tray slot and location information of slides accommodated in the slide trays. In an embodiment, the slide scanning apparatus 100 may identify whether slides are accommodated in the slide tray, positions of slides accommodated in the slide tray, and a number of slides accommodated in the slide tray.

In an embodiment, the slide scanning apparatus 100 may identify one or more slides arranged in each slide tray and, based on the identified one or more slides, may generate the second GUI 520 regarding slides. For example, the second GUI 520 may comprise a GUI indicating location information of the one or more slides in the slide tray, slide scan progress information regarding at least a portion of the one or more slides, specimen information of the one or more slides, code information of the one or more slides, identification information of the slide tray, and identification information of the one or more slides.

In an embodiment, the second GUI 520 may comprise: a GUI 522 corresponding to a first slide for which scanning has been completed; a GUI 524 corresponding to a second slide being scanned; a GUI 526 corresponding to a third slide for which scanning has not yet been performed; a GUI 528 indicating that no slide is present in a slide accommodation portion; and a GUI 529 indicating a slide that is not a scan target. In addition, the GUI 524 corresponding to the second slide being scanned may comprise a GUI indicating a scanning progress degree of the second slide. For example, when scanning of the second slide has proceeded to 33%, the GUI 524 corresponding to the second slide may be configured to indicate that the scanning has progressed to 33%.

In an embodiment, the second GUI indicating location information of the one or more slides in the slide tray may correspond to actual locations of slides located in the slide tray. For example, when a slide tray comprises two rows of slide accommodation portions, each row accommodating eight slides, the slide scanning apparatus 100 may generate and provide the second GUI having GUIs corresponding to slide accommodation portions arranged in two rows and eight columns. In addition, as illustrated in FIG. 6, when only eight slides in a first row and two slides on a left side in a second row are present among slides of two rows and eight columns in an actual slide tray, the slide scanning apparatus 100 may generate and provide slide-shaped GUIs at positions corresponding to the first row of eight slide accommodation portions and the left two slide accommodation portions in the second row among the GUIs corresponding to the slide accommodation portions of two rows and eight columns. According to an embodiment, a UI/UX may be provided that allows a user to intuitively recognize locations of slides on the slide tray.

In an embodiment, the slide scan progress information regarding at least a portion of the one or more slides may comprise scan progress information regarding slides for which scanning has been completed or is in progress among the one or more slides identified as being present in the slide tray. Referring to FIG. 6 as an example, based on the fact that slide scanning has been completed for slides having identification numbers 1 to 3, the slide scanning apparatus 100 may provide a GUI indicating that scanning of the slides having identification numbers 1 to 3 has been completed. For example, the slide scanning apparatus 100 may provide a GUI including a phrase indicating that scanning has been completed, or may provide GUIs by configuring a GUI corresponding to a slide for which scanning has been completed in a different color from a GUI corresponding to a slide for which scanning has not been completed (for example, slides having identification numbers 5 to 8). For example, the slide scanning apparatus 100 may generate GUIs corresponding to slides for which scanning has been completed or is in progress in chromatic colors, and may generate GUIs corresponding to slides scheduled to be scanned in achromatic colors. In an embodiment, based on the fact that scanning of a slide having identification number 4 is in progress, the slide scanning apparatus 100 may provide a GUI 524 including scan progress information of the slide having identification number 4. The scan progress information regarding the slide may comprise information indicating a scan progress degree and whether scanning is in progress.

In an embodiment, the slide scanning apparatus 100 may generate the second GUI indicating location information of the one or more slides in the slide tray, slide scan progress information regarding at least a portion of the one or more slides, specimen information of the one or more slides, code information of the one or more slides, identification information of the slide tray, and identification information of the one or more slides.

In an embodiment, the slide scanning apparatus 100 may obtain a first user input for selecting a first slide tray among one or more slide trays based on the first GUI 510. In addition, based on the first user input, the slide scanning apparatus 100 may control the display 120 to output the second GUI 520 corresponding to the first slide tray and including identification information 540 of the first slide tray. The identification information 540 of the first slide tray may be information regarding the slide tray selected by the user through the first GUI 510 and may comprise location information indicating a location of the first slide tray in the slide tray slot, scan status information of the first slide tray, information regarding the number of slides included in the first slide tray, and information indicating a time when the first slide tray was inserted into the slide tray slot.

In an embodiment, the location information indicating the location of the first slide tray in the slide tray slot may comprise information indicating which position in a stack of slide tray accommodation units the slide tray occupies when the slide tray accommodation units are arranged in a stack in the slide tray slot. For example, when the first slide tray is inserted into a slide tray accommodation unit that is fifth from the top of the slide tray slot, when the slide scanning apparatus 100 receives a user input corresponding to the slide tray that is fifth from the top through the first GUI 510, the slide scanning apparatus 100 may control the display to output a phrase “05 Slot-Tray” through the second GUI 520.

In an embodiment, the scan status information may comprise information regarding a scan-progressed time, an estimated time remaining until completion of scanning, a number of slides scanned, a number of slides for which scanning has not yet been completed, a scan magnification, and a scan performer.

In an embodiment, when a slide tray is inserted into a slide tray accommodation unit in the slide tray slot, the slide tray slot may store an insertion time and may transmit the insertion time information to the slide scanning apparatus 100. The slide scanning apparatus 100 may control the display 120 to output information indicating a time when the slide tray was inserted into the slide tray slot based on the time information received from the slide tray slot.

In an embodiment, the slide scanning apparatus 100 may control the display 120 to output the third GUI 530 together with the second GUI 520. When the slide tray corresponding to information currently displayed through the second GUI 520 is a first slide tray, the third GUI 530 may comprise a GUI for selecting a second slide tray that is not the first slide tray. For example, the third GUI 530 may comprise a GUI for selecting a slide tray located before or after the first slide tray.

In an embodiment, while outputting the second GUI 520 regarding the first slide tray, the slide scanning apparatus 100 may obtain a second user input for selecting a second slide tray that is not the first slide tray. The second user input may comprise a user input of touching a GUI corresponding to the second slide tray in the first GUI 510, a user input of touching a GUI corresponding to the second slide tray in the third GUI 530, or a gesture input of dragging the display from one direction to another direction. In addition, based on the second user input, the slide scanning apparatus 100 may update at least one of the first GUI, the second GUI, and the third GUI. For example, before the second user input is obtained, the first GUI 510 may comprise a fourth GUI 550 corresponding to the first slide tray (05 Slot-Tray in FIG. 6) and a fifth GUI 570 corresponding to the second slide tray (04 Slot-Tray in FIG. 6, which is a previous slot of 05 Slot-Tray), and since information currently displayed through the second GUI 520 is information regarding the first slide tray, the fifth GUI 570 may be formed smaller than the fourth GUI 550. For example, when information currently displayed through the second GUI 520 corresponds to the 05 Slot-Tray, the fourth GUI 550 may be formed larger than the fifth GUI 570. In this case, the slide scanning apparatus 100 may obtain a user input for selecting the 04 Slot-Tray, which is a previous tray of the 05 Slot-Tray, through the third GUI 530. Accordingly, the slide scanning apparatus 100 may update the second GUI 520 such that information displayed through the second GUI 520 corresponds to the second slide tray (04 Slot-Tray), and may update the first GUI 510 such that the fifth GUI 570 corresponding to the second slide tray (04 Slot-Tray) is formed larger than the fourth GUI 550 corresponding to the first slide tray (05 Slot-Tray). As a result, the fourth GUI 550 corresponding to the first slide tray (05 Slot-Tray) may have the same size as GUIs corresponding to slide trays other than the second slide tray (04 Slot-Tray).

According to an embodiment of the present disclosure, various functions for providing slide information may be intuitively provided by allowing a user to modify a region of interest (ROI) through a user input such as a touch input.

In an embodiment, the slide scanning apparatus 100 may identify a color corresponding to each slide tray (or slide tray accommodation unit) in the first GUI 510 and may transmit, to the slide tray slot, a signal for controlling light emitting elements of the respective slide tray accommodation units of the slide tray slot to emit light corresponding to the colors associated with the respective slide trays in the first GUI 510. Alternatively, the slide scanning apparatus 100 may receive, from the slide tray slot, color information of light emitted from light emitting elements of the respective slide tray accommodation units of the slide tray slot and, based on the color information, may control the display 120 to output the first GUI 510 corresponding to a slide tray in each slide tray accommodation unit in a color corresponding to the color of light emitted from a light emitting element of each slide tray accommodation unit.

According to an embodiment of the present disclosure, an intuitive UI/UX may be provided to a user by synchronizing colors of light emitting elements of the respective slide tray accommodation units in an actual slide tray slot with colors of GUIs provided by the slide scanning apparatus 100.

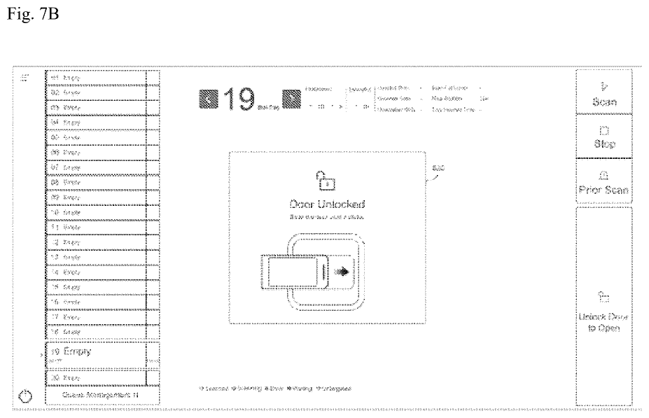

In an embodiment, the slide scanning apparatus 100 may output a UI 600 for opening the door of the slide scanning apparatus 100 to the display 120. When the slide scanning apparatus 100 obtains a user touch input clicking the UI 600 for opening the door, the slide scanning apparatus 100 may control the display to provide information regarding an open state of the door. Related descriptions will be provided in further detail with reference to FIG. 7.

FIG. 7 is a view illustrating a UI/UX in an open state of the door according to an embodiment of the present disclosure. FIG. 7A illustrates a UI/UX when the door is in a section in which the door changes from the closed state to the fully-open state, and FIG. 7B illustrates a UI/UX when the door is in a section in which the door changes from the fully-open state to the closed state.

Referring to FIG. 7, when the door is opened, the slide scanning apparatus 100 may identify a degree to which the door is moved and may output, to the display 120, a UI indicating the degree to which the door has been moved. Since the door can be locked when opened to the fully-open state, stability in the use of the slide scanning apparatus 100 can be ensured. Accordingly, when the door is opened, the slide scanning apparatus 100 may provide a door opening state-related interface 610 or 620, may identify whether the door has moved to a specific position, and may control the display 120 such that, when it is identified that the door has moved to the specific position, that is, when the door is in the fully-open state, the door opening state-related interface 610 is not provided. Similarly, in a closing process in which the door changes from the fully-open state to a locked state, a related interface 620 may be provided in a similar manner. According to an embodiment, a user may move the door until a screen of the interface is changed through the door opening state-related interface 610 output to the display 120. Accordingly, when the user opens or closes the door, the user may check, through the UI/UX, whether the door has been properly opened or closed, thereby increasing stability in the use of the slide scanning apparatus 100 and also improving user convenience in using the slide scanning apparatus 100.

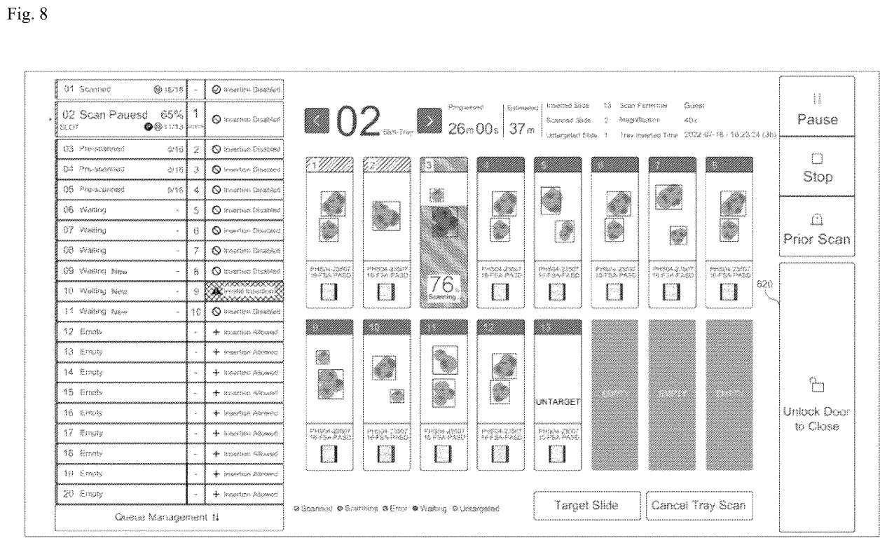

In an embodiment, when the door reaches the fully-open state, the slide scanning apparatus 100 may provide an interface as illustrated in FIG. 8.

FIG. 8 is a view illustrating a UI/UX in a fully-open state of the door according to an embodiment of the present disclosure.

Referring to FIG. 8, when the door is in the fully-open state or the open state, operation of equipment inside the slide scanning apparatus 100 may compromise safety. Therefore, when the door is in the fully-open state or the open state, the slide scanning apparatus 100 may control the apparatus such that a slide scanning operation is not performed. Accordingly, when the door is in the fully-open state or the open state, the slide scanning apparatus 100 may display information implying that slide scanning operations are not possible. That is, when the door is in the open state and/or the fully-open state, the slide scanning apparatus 100 may not obtain a user touch input to a specific region of the display. The specific region may include a region for starting a scanning process. For example, as illustrated in FIG. 8, the slide scanning apparatus 100 may provide a UI having a darker shade than the UI of FIG. 6 so as to imply that scanning does not proceed even if a UI regarding slide scanning is touched. In addition, the slide scanning apparatus 100 may be designed such that only a UI 600 for closing the door is enabled to accept touch input and may output the UI 600 to the display 120.

In an embodiment, when the door is in the closed state, touch inputs to UIs regarding scan operations such as scan start, scan stop, or scan order setting may be obtained, and when the door is in the open state or the fully-open state, the UIs may be configured such that the above scan operation settings are not possible. Furthermore, when the door is in the fully-open state, a UI may be set such that insertion or removal of a tray is possible, or slot status information regarding a slide tray slot related to insertion or removal of a tray may be displayed. Information regarding slide trays, information regarding display of slide scan progress state, and the like may be provided regardless of the state of the door.

According to an embodiment of the present disclosure, stability of the apparatus may be improved by configuring an interface such that internal operations related to slide scanning are not possible when the door is in the open state or the fully-open state.

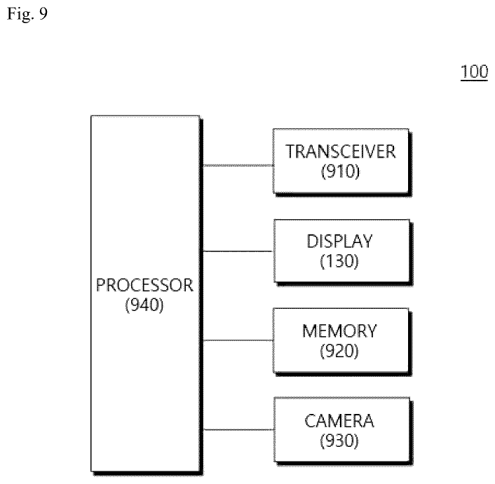

FIG. 9 is a block diagram of a slide scanning apparatus according to an embodiment of the present disclosure.

Referring to FIG. 9, the slide scanning apparatus 100 may comprise a transceiver 910, the display 120, a memory 920, a camera 930, and a processor 940. However, not all components illustrated in FIG. 9 are essential components of the slide scanning apparatus 100. The slide scanning apparatus 100 may be implemented with more components than those illustrated in FIG. 9 or may be implemented with fewer components than those illustrated in FIG. 9. For example, the slide scanning apparatus 100 may further comprise a slide tray slot, a sensor unit, and a speaker (not shown). In addition, the transceiver 910, the display 120, the memory 920, the camera 930, and the processor 940 may be implemented in the form of a single chip.

In an embodiment, the transceiver 910 may communicate with a terminal or another electronic device that is connected to the slide scanning apparatus 100 via a wired or wireless connection. For example, the slide scanning apparatus 100 may receive, from the slide tray slot, information regarding whether a slide tray is mounted in each slide tray accommodation unit.

The display 120 may comprise a touch panel, a hologram device, a projector, a screen, or a monitor. In addition, a key pad, an ultrasonic input device, a pen sensor, or the like may be further provided together with the display 120.

Various types of data such as programs (for example, applications) and files may be installed and stored in the memory 920. The processor 940 may access data stored in the memory 920 and use the data, or may store new data in the memory 920. In an embodiment, the memory 920 may comprise a database.

The camera 930 may obtain a preview image of a slide tray and individual scan images of slides located in the slide tray.

The processor 940 controls overall operations of the slide scanning apparatus 100 and may comprise at least one processor such as a CPU or a GPU. The processor 940 may control other components included in the slide scanning apparatus 100 to perform operations for operating the slide scanning apparatus 100. For example, the processor 940 may execute programs stored in the memory 920, may read stored files, or may store new files. In an embodiment, the processor 940 may perform operations for operating the slide scanning apparatus 100 by executing programs stored in the memory 920. For example, the processor 940 may identify a door state comprising at least one of a closed state of the door, an open state of the door, or a fully-open state of the door, and may control the display 120 to output a user interface corresponding to the door state based on the door state.

In an embodiment, the slide scanning apparatus 100 may comprise a slide tray slot inside the door. When the door is in the open state or the fully-open state, the slide scanning apparatus 100 may identify that a slide tray is inserted into the slide tray slot and may provide a first GUI regarding one or more slide trays located in the slide tray slot and a second GUI regarding one or more slides located in a slide tray. Here, the first GUI and the second GUI may include one or more GUIs that can receive touch input when the door is in the closed state, and may be provided such that touch input to the GUIs cannot be received when the door is in at least one of the open state and the fully-open state.

An embodiment of the present disclosure may be implemented in the form of a recording medium including computer-executable instructions, such as program modules executed by a computer. The computer-readable medium may be any available medium accessible by a computer and may include both volatile and non-volatile media and both removable and non-removable media. The computer-readable medium may also include computer storage media and communication media. Computer storage media may include volatile and non-volatile, removable and non-removable media implemented in any method or technology for storage of information such as computer-readable instructions, data structures, program modules, or other data. Communication media typically include computer-readable instructions, data structures, or program modules and include any information delivery media.

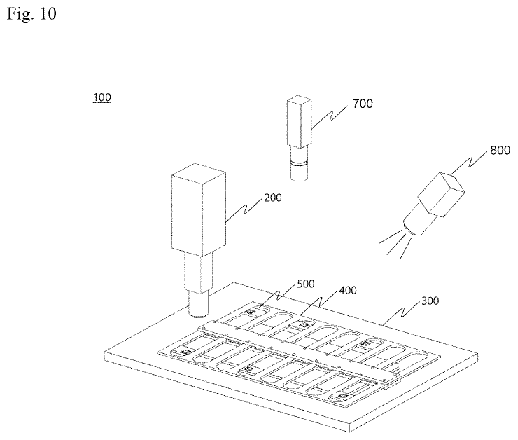

FIG. 10 is a view illustrating a slide scanning apparatus according to an embodiment of the present disclosure.

A slide scanning apparatus 100 according to an embodiment of the present disclosure may obtain a preview image of a slide tray, recognize information regarding a slide tray, slides, or pathological samples from the preview image, obtain a slide scan image based on the recognized information, and store the slide scan image in association with information regarding the slide tray, the slides, or the pathological samples.

In an embodiment, the slide scanning apparatus 100 may comprise a preview camera 700, a scan camera 200, and a slide tray stage 300. For example, in a state in which a slide tray 400 is mounted on the slide tray stage 300, a preview image of a plurality of slides 500 may be obtained using the preview camera 700, and images of pathological samples accommodated in the respective slides 500 may be obtained using the scan camera 200.

In an embodiment, the slide tray stage 300 may transport the slide tray 400 to a position at which a preview image can be obtained by the preview camera 700 and, after the preview image has been obtained, may transport the slide tray 400 again to a position at which an image of a pathological sample can be obtained by the scan camera 200. In another embodiment, a first slide tray stage 300 may transport the slide tray 400 to a position at which a preview image can be obtained by the preview camera 700, and after the preview image has been obtained, a second slide tray stage (not shown), which is separate from the first slide tray stage 300, may transport the slide tray 400 to a position at which an image of a pathological sample can be obtained by the scan camera 200.

In an embodiment, the preview camera 700 may obtain a preview image of the plurality of slides 500 mounted on the slide tray stage 300. For example, the preview camera 700 may obtain an entire image of the slide tray 400 as a preview image. In another example, the preview camera 700 may obtain, as a preview image, an image of a portion of the slides 500 mounted on the slide tray 400.

Referring to the embodiment of FIG. 10, the preview camera 700 may be disposed at a predetermined distance from the slide tray stage 300 so as to obtain a preview image of the plurality of slides 500. The preview camera 700 may have a resolution suitable for obtaining information to be derived from the preview image. For example, the preview camera 700 may be configured as a camera having a lower resolution than the scan camera 200.

In an embodiment, the preview camera 700 may comprise one or more lenses, an image sensor, and a focus controller. The preview camera 700 may adjust a focus on the slide tray by using the focus controller and may obtain an image, which has passed through the one or more lenses, by the image sensor.

In an embodiment, the scan camera 200 may obtain a scan image of a pathological sample included in each slide 500. The scan camera 200 may divide one slide 500 into a plurality of regions, obtain scan images for the respective regions, and then combine the scan images to obtain an image of the entire slide 500. For example, the scan camera 200 may divide a region of the slide 500 into rectangular tiles and obtain scan images for the respective regions. In another example, the scan camera 200 may divide a slide region into a plurality of lines, obtain scan images in a line scan manner, and then combine the scan images. In another example, the scan camera 200 may obtain an image of an entire slide at one time. However, a method by which the scan camera 200 obtains an image of the slide 500 is not limited to the above embodiments.

In an embodiment, the scan camera 200 may comprise one or more lenses, an image sensor, and a focus controller. The scan camera 200 may adjust a focus on each region of a slide to be scanned by using the focus controller and may obtain an image, which has passed through the one or more lenses, by the image sensor.

In an embodiment, the slide tray stage 300 may support the slide tray 400 on an upper surface thereof. In an embodiment, the slide tray stage 300 may comprise a fixing means (not shown) configured to fix the slide tray 400 on the upper surface thereof. For example, the slide tray stage 300 may comprise a means for fixing all or part of the slide tray 400 in a vertical direction or a horizontal direction.

In an embodiment, the slide tray stage 300 may move the slide tray 400 to a position at which the preview camera 700 or the scan camera 200 can obtain an image. For example, the slide tray stage 300 may move the slide tray 400 to a predetermined position at which the preview camera 700 can obtain an entire image of the slide tray 400. In addition, the slide tray stage 300 may move the slide tray 400 to a predetermined position such that the scan camera 200 can scan a region of the slide 500. Furthermore, after the scan camera 200 has completed scanning for one region, the slide tray stage 300 may move the slide tray 400 such that a next region can be scanned.

In another embodiment, the first slide tray stage 300 may move the slide tray 400 to a position at which the preview camera 700 or the scan camera 200 can obtain an image. For example, the first slide tray stage 300 may move the slide tray 400 to a predetermined position at which the preview camera 700 can obtain an entire image of the slide tray 400. Thereafter, a second slide tray stage (not shown and separate from the first slide tray stage 300) may move the slide tray 400 to a predetermined position such that the scan camera 200 can scan a region of the slide 500. In addition, the second slide tray stage (not shown) may move the slide tray 400 such that a next region can be scanned after the scan camera 200 has completed scanning for one region.

In an embodiment, when the scan camera 200 uses a line scan method, the slide tray stage 300 may move the slide tray 400 for line scanning. For example, the slide tray stage 300 may move the slide tray 400 in accordance with a line scan speed of the scan camera 200.

In another embodiment, the slide tray stage 300 may not move, and the preview camera 700 or the scan camera 200 may move to a predetermined position to obtain a preview image or a slide image. For example, the slide tray stage 300 may be fixed in position, and the preview camera 700 or the scan camera 200 may be mounted on a movable arm and moved to a position for obtaining a preview image or a slide scan image.

In an embodiment, the slide scanning apparatus 100 may further comprise an illumination unit 800 configured to provide illumination to the slide tray 400 and/or the slides 500. Referring to the embodiment of FIG. 10, the illumination unit 800 may provide illumination from an upper surface of the slide tray 400. In another embodiment, the illumination unit 800 may provide illumination from a lower surface of the slide tray 400. In still another embodiment, the illumination unit 800 may provide illumination to both the upper surface and the lower surface of the slide tray 400.

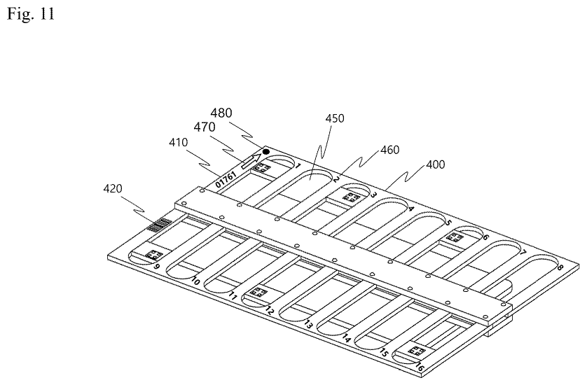

FIG. 11 is a view illustrating a slide tray according to an embodiment of the present disclosure.

The slide scanning apparatus 100 may recognize information regarding a slide tray 400 from a preview image obtained through the preview camera 700.

Referring to FIG. 11, the slide tray 400 may comprise a plurality of slide accommodation portions 450, each of which can accommodate a slide, and may comprise identification information 410 and 420 regarding the slide tray 400, direction indication information 470 and 480 regarding a direction of the slide tray 400, and identification information 460 regarding each slide accommodation portion. For example, the identification information 410 and 420 regarding the slide tray 400 may be printed as letters or numbers, or may be included in the form of a barcode or a QR code. When the identification information 410 and 420 regarding the slide tray 400 is printed as letters or numbers, the identification information may be identified from the preview image by using an optical character recognition (OCR) technique, and when the identification information is in the form of a barcode or a QR code, the identification information regarding the slide tray 400 may be obtained by recognizing the barcode or the QR code. The slide scanning apparatus 100 may recognize the identification information 410 and 420 regarding the slide tray 400 and may accurately identify the slide tray 400 currently being scanned.

In an embodiment, the slide scanning apparatus 100 may identify, from the preview image, whether the slide tray 400 is located on the slide tray stage 300. For example, a marker may be placed at a predetermined position on the slide tray stage 300 such that the marker is placed at a position where the marker is obscured when the slide tray 400 is placed on the slide tray stage 300, and the presence of the slide tray 400 may be simply determined in the preview image according to whether the marker is recognized. Furthermore, if the marker is arranged such that the marker is obscured only when the slide tray 400 is placed correctly, it can also be determined, according to whether the marker is recognized in the preview image, whether the slide tray 400 is placed in a correct position. In another example, the slide scanning apparatus 100 may determine the presence of the slide tray 400 in the preview image according to whether the identification information 410 and 420 regarding the slide tray 400 or the direction indication information 470 and 480 regarding the slide tray 400 is recognized.

In an embodiment, the direction indication information 470 and 480 regarding the slide tray 400 may comprise an indication of an up-down direction or a left-right direction, and may be displayed as an arrow indicating a direction or as a predetermined identifier on one side of a specific direction. For example, referring to FIG. 11, the direction indication information 470 regarding the slide tray 400 may be displayed by an arrow representing an up-down direction of the slide tray 400. In another example, a predetermined identifier (for example, a black dot in FIG. 11) may be displayed at an upper left corner of the slide tray 400 to display the direction indication information 480 regarding both the up-down direction and the left-right direction. The slide scanning apparatus 100 may recognize the direction indication information 470 and 480 regarding the slide tray 400 by recognizing a direction of the arrow in the preview image or recognizing a specific identifier at a predetermined position. In an embodiment, the slide scanning apparatus 100 may recognize a direction in which the slide tray 400 is placed on the slide tray stage 300 by using the direction indication information 470 and 480 regarding the slide tray 400.

In an embodiment, the identification information 460 regarding each slide accommodation portion 450 of the slide tray 400 may be displayed as numbers, a number of dots, a length of a line, or the like. For example, the identification information 460 regarding each slide accommodation portion 450 may be displayed as numbers, and the slide scanning apparatus 100 may recognize the identification information 460 regarding each slide accommodation portion 450 from the preview image by using an OCR technique. In another example, when the identification information 460 regarding each slide accommodation portion 450 is expressed as a predetermined symbol such as a number of dots or a length of a line, the slide scanning apparatus 100 may recognize the symbol from the preview image and may recognize the identification information regarding each slide accommodation portion 450.

In an embodiment, when a specific slide 500 accommodated in the slide tray 400 is broken or has been dislodged from a slide accommodation portion 450 in the preview image, the slide scanning apparatus 100 may recognize the corresponding slide accommodation portion 450 by using the identification information 460. For example, when a fourth slide from the left in an upper slide row is broken, the slide scanning apparatus 100 may recognize the identification information 460 of the corresponding slide accommodation portion 450 and may recognize that a slide at position 4 is broken.

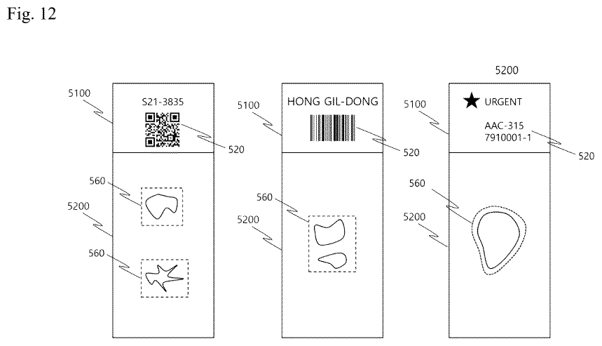

FIG. 12 is a view illustrating a slide according to an embodiment of the present disclosure.

The slide scanning apparatus 100 may recognize information regarding each slide 500 accommodated in the slide tray 400 from a preview image obtained through the preview camera 700.

Referring to FIG. 12, the slide 500 may comprise an information inscription portion 5100 for displaying information regarding the slide 500 and a sample accommodating portion 5200 for accommodating a pathological sample. The information inscription portion 5100 of the slide 500 may display information regarding the slide 500 by using letters, numbers, symbols, a barcode, a QR code, or the like. For example, when the information is displayed as letters and/or numbers, the information may be recognized by using an OCR technique, when the information is expressed as a predetermined symbol, the information may be recognized by using a camera vision technique, and when the information is displayed as a QR code or a barcode, the information may be recognized by decoding the QR code or the barcode.

In an embodiment, the information inscription portion 5100 of the slide 500 may include information regarding a patient corresponding to the slide 500, information regarding a pathological sample, and information regarding a scan method. For example, the information regarding a patient corresponding to the slide 500 may comprise a patient name, a patient age, a national identification number of the patient, a patient ID (or patient number), a medical department, and a disease name. The information regarding the pathological sample may comprise a specimen site of the corresponding sample (for example, liver tissue), whether staining has been performed, a name of a staining reagent, and a specimen collection time. In addition, the information regarding the scan method may comprise information regarding a priority of the sample (for example, whether the sample is for an urgent examination), a scan resolution, a scan region, and a scan method.

In an embodiment, the slide scanning apparatus 100 may recognize a position of a pathological sample on the slide 500 in the preview image and may previously identify a region of interest (ROI) to be scanned by the scan camera 200. For example, the slide scanning apparatus 100 may recognize a position of a pathological sample in the sample accommodating portion 5200 of the slide 500, store the position, and then cause the scan camera 200 to scan the region of interest so as to obtain an image of the pathological sample. Referring to FIG. 12, the region of interest may be set as a rectangle including the pathological sample according to a scan method, or may be set along an outline of the pathological sample.

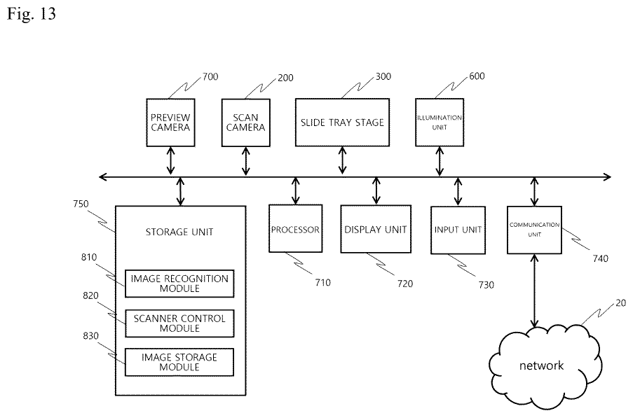

FIG. 13 is a view illustrating a slide scanning apparatus according to an embodiment of the present disclosure.

In an embodiment, the slide scanning apparatus 100 may comprise a processor 710, a display unit 720, an input unit 730, a communication unit 740, and a storage unit 750 in addition to the preview camera 700, the scan camera 200, the slide tray stage 300, and the illumination unit 800. However, not all of components illustrated in FIG. 13 are essential components of the slide scanning apparatus 10.

In an embodiment, the processor 710 may be configured as a general-purpose processor or a dedicated processor. The processor 710 may read program modules stored in the storage unit 750 and execute the program modules. For example, by executing the program modules stored in the storage unit 750, the processor 710 may control the preview camera 700, the scan camera 200, the slide tray stage 300, or the illumination unit 800. In addition, by executing the program modules stored in the storage unit 750, the processor 710 may output, to the display unit 720, a current state of the slide scanning apparatus 10, a progress status of slide scanning, and/or a slide image for which scanning has been completed. Furthermore, by executing the program modules stored in the storage unit 750, the processor 710 may transmit and receive information to and from an external device through the communication unit 740. In addition, by executing the program modules stored in the storage unit 750, the processor 710 may receive a user input from the input unit 730. In an embodiment, the input unit 730 may be configured as a touch screen on the display unit 720.

In an embodiment, an image recognition module 810, a scanner control module 820, and an image storage module 830, which are program modules, may be stored in the storage unit 750 and may be executed through the processor 710. For example, the storage unit 750 may comprise a memory, an SSD, an HDD, or a combination thereof.

In an embodiment, the image recognition module 810 may recognize, from a preview image obtained through the preview camera 700, information regarding the slide tray 400, the slides 500, and/or pathological samples. For example, the image recognition module 810 may distinguish a slide tray region, slide regions, and an information inscription portion 5100 and a sample accommodating portion 5200 of a slide region, may extract information regarding the slide tray 400 from the slide tray region, may extract information regarding a slide from each slide region, and may recognize information regarding a corresponding pathological sample from the information inscription portion 5100 and the sample accommodating portion 5200 of each slide region.

In an embodiment, the image recognition module 810 may recognize, from the preview image, information regarding the slide tray 400. Here, the information regarding the slide tray 400 may comprise whether the slide tray 400 is present on the slide tray stage 300, identification information regarding the slide tray 400, direction information regarding the slide tray 400, or identification information 460 regarding each slide accommodation portion 450 of the slide tray 400. For example, whether the slide tray 400 is present on the slide tray stage 300 may be determined according to whether a marker located on an upper surface of the slide tray stage 300 (which is obscured when the slide tray 400 is placed in a correct position) is recognized. For example, the identification information regarding the slide tray 400, the direction information regarding the slide tray 400, or the identification information 460 regarding each slide accommodation portion 450 of the slide tray 400 may be recognized by letters, numbers, symbols, a barcode, or a QR code displayed on an upper surface of the slide tray 400.

In an embodiment, the image recognition module 810 may recognize, from the preview image, information regarding the slides 500. Here, the information regarding the slides 500 may comprise whether a slide 500 is present in each slide accommodation portion 450, whether the slide 500 in each slide accommodation portion 450 is broken, whether the slide 500 is properly accommodated in each slide accommodation portion 450, or information regarding a region of interest in which a pathological sample is present in the slide 500. For example, whether the slide 500 is present, whether the slide 500 is broken, and whether the slide 500 is in a correct position in each slide accommodation portion 450 may be obtained through analysis of the preview image, and, for convenience of image analysis, a specific marker may be added at a predetermined position of each slide accommodation portion and/or each slide. For example, information regarding a region of interest in which a pathological sample is present in the slide 500 may be set as a region of interest including the pathological sample by recognizing an outline of the pathological sample in the sample accommodating portion 5200 of each slide 500.

In an embodiment, the image recognition module 810 may recognize, from the preview image, information regarding a pathological sample. Here, the information regarding the pathological sample may comprise a specimen site of the pathological sample, a specimen collection time, whether staining has been performed, a name of a staining reagent, or patient information corresponding to the pathological sample. In addition, the patient information corresponding to the pathological sample may comprise a patient name, a patient age, a national identification number of the patient, a patient number, a medical department of the patient, or a disease name of the patient. In an embodiment, the information regarding the pathological sample may comprise information regarding a priority of the pathological sample, a scan resolution, a scan region, or a scan method. For example, information regarding a pathological tissue (or a pathological sample) may be obtained by recognizing letters, numbers, symbols, a barcode, or a QR code written in the information inscription portion 5100 of each slide 500.

In an embodiment, the scanner control module 820 may control the preview camera 700 to obtain a preview image and may control the slide scanning apparatus 100 by using information regarding the slide tray 400, the slides 500, and/or pathological samples recognized by the image recognition module 810 so as to obtain scan images of pathological samples. For example, the scanner control module 820 may control the scan camera 200, the slide tray stage 300, and the illumination unit 800 to obtain a scan image by using information regarding the slide tray 400, the slides 500, and/or the pathological samples.

In an embodiment, the scanner control module 820 may determine a lens magnification of the scan camera 200, a scan resolution, a size of a scan region, a scan method, an illumination intensity of the illumination unit 800, an illumination method, and the like by considering a disease name of a patient corresponding to a pathological sample, a tissue name of a body part of the pathological sample, whether staining has been performed, a name of a staining reagent, and a specimen collection time, and may control the slide scanning apparatus 100 based on the determined parameters. In another embodiment, the scanner control module 820 may control the slide scanning apparatus 100 based on information regarding a scan method (for example, priority, scan resolution, scan region, scan method, and the like) recognized by the image recognition module 810 from the information inscription portion 5100 of the slide 500 in the preview image.

In an embodiment, the scanner control module 820 may provide a preview image together with information regarding the slide tray 400, the slides 500, and/or pathological samples to a user and may receive a selection regarding slide scanning from the user. In this case, the scanner control module 820 may control slide scanning based on the user's selection. For example, the user may select to perform scanning only for some slides accommodated in the slide tray. In another example, the user may select to perform scanning with priority for a specific slide accommodated in the slide tray. In another example, the user may select a scan method for a specific slide.

In an embodiment, the image storage module 830 may store a scan image obtained by the scanner control module 820 in association with information regarding a pathological sample obtained by the image recognition module 810. For example, the image storage module 830 may store the scan image by including, as macro information, a patient name, a patient age, a national identification number of the patient, a patient ID (or patient number), a medical department, a disease name, and the like obtained by the image recognition module 810. For example, the image storage module 830 may store the scan image by including, as macro information, a tissue name of a body part of the sample (for example, liver tissue), whether staining has been performed, a name of a staining reagent, and a specimen collection time obtained by the image recognition module 810. For example, the image storage module 830 may store the scan image by including, as macro information, a priority (for example, whether the sample is for an urgent examination), a scan resolution, a scan region, and a scan method obtained by the image recognition module 810.

In an embodiment, the image storage module 830 may store the scan image by including, as macro information, a scan resolution, a scan region, a scan method, and the like used in a process of obtaining the scan image in the scanner control module 820.

In an embodiment, the image storage module 830 may transmit, to an external device by using the communication unit 740, a scan image obtained by the scanner control module 820 based on information regarding a pathological sample obtained by the image recognition module 810. For example, when, according to the information regarding the pathological sample, the pathological sample is a target of an urgent examination, the image storage module 830 may transmit a scan image regarding the pathological sample to the external device in association with the information regarding the pathological sample. As a result, a medical professional who has requested an urgent examination may be provided with the image regarding the pathological sample with priority.

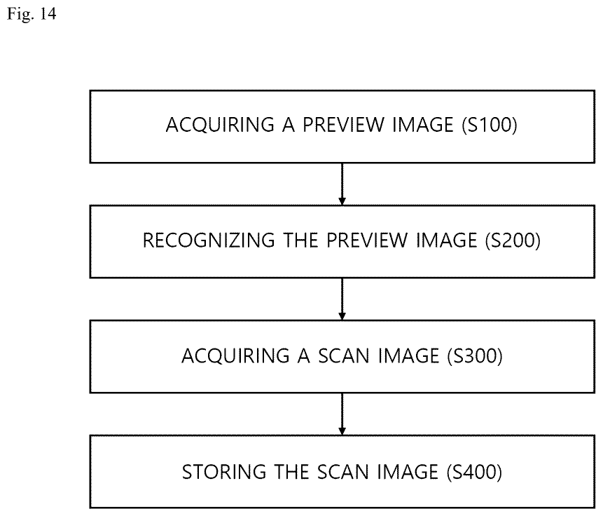

FIG. 14 illustrates a slide scanning method according to an embodiment of the present disclosure.

According to an embodiment of the present disclosure, the slide scanning method may comprise: acquiring preview image (S100); recognizing preview image (S200); acquiring scan image (S300); and storing scan image (S400).

In an embodiment, the step of acquiring preview image (S100) is a process of obtaining a preview image of the slide tray using the preview camera 700. For example, the slide tray stage 300 may be moved to a position where the preview camera 700 can acquire a preview image of the entire slide tray, if necessary, the illumination unit 800 is used to provide the required illumination, and the preview camera 700 may be operated to obtain the preview image of the slide tray 400.

In an embodiment, when the preview image does not satisfy a predetermined criterion, the preview camera 700, the slide tray stage 300, or the illumination unit 800 may be controlled to acquire the preview image again. For example, when the quality of the preview image is below a threshold, the preview camera 700 may reacquire the preview image. As another example, when a portion of the slide tray is not captured in the preview image, the slide tray stage 300 may be adjusted and the preview image may be reacquired.

In an embodiment, the step of recognizing preview image (S200) is a process of recognizing necessary information from the preview image.

In the step of recognizing preview image (S200) according to an embodiment, information regarding accommodation states of the slide tray 400 and the slides 500 may be obtained. First, the identification information 410 and 420 of the slide tray 400 and the direction indication information 470 and 480 may be recognized to identify the type of slide tray 400 and the direction in which the slide tray 400 is placed on the slide tray stage 300. Next, whether a slide 500 exists in each slide accommodation portion 450, whether the slide 500 is damaged, and whether the slide 500 is properly positioned may be recognized. When a slide 500 is missing, damaged, detached, or improperly accommodated, information regarding the corresponding slide accommodation portion 450 may be stored using its position and/or identification information 460. For example, among slide accommodation portions 1 through 16, it may be recognized that slides are not present in slide accommodation portions 2, 6, and 11, that a slide in slide accommodation portion 13 is damaged, and that a slide in slide accommodation portion 7 is partially dislodged from the slide accommodation portion.

In the step of recognizing preview image (S200) according to an embodiment, information regarding each slide 500 accommodated in the slide accommodation portions 450 may be recognized. Characters, numbers, symbols, barcodes, or QR codes written in the information inscription portion 5100 of each slide 500 may be recognized to obtain information regarding a patient, a pathological sample, or a scanning method.

In an embodiment, the step of acquiring scan image (S300) may obtain scan images of pathological samples by controlling the slide scanning apparatus 100 based on the information regarding the slide tray 400, the slides 500, and/or the pathological samples recognized in the step of recognizing preview image (S200). For example, the scanner control module 820 may acquire scan images by controlling the scan camera 200, the slide tray stage 300, and the illumination unit 800 using the information regarding the slide tray 400, the slides 500, and/or the pathological samples.

In an embodiment, the step of acquiring scan image (S300) may determine the lens magnification, scan resolution, scan region size, scan method, illumination intensity, and illumination mode of the scan camera 200, considering the disease of the patient corresponding to the pathological sample, the type of tissue, whether staining has been performed, a name of a staining reagent, and sample collection time, and may control the slide scanning device 10 based thereon. In another embodiment, the step of acquiring scan image (S300) may control the slide scanning apparatus 100 based on information regarding a scan method (e.g., priority, scan resolution, scan region, scan method, etc.) recognized from the slide's information inscription portion 5100 in the step of recognizing preview image (S200).

In an embodiment, before acquiring scan image (S300), the preview image together with information regarding the slide tray 400, the slides 500, and/or the pathological samples may be provided to a user, and a selection regarding slide scanning may be received from the user. In such a case, the step of acquiring scan image (S300) may be performed based on the user's selection. For example, the user may select to perform scanning only for some slides accommodated in the slide tray. In another example, the user may select to perform scanning with priority for a specific slide accommodated in the slide tray. In another example, the user may select a scan method for a specific slide.

In an embodiment, the step of storing scan image (S400) may store the scan image obtained in the step of acquiring scan image (S300) in association with information regarding the pathological sample obtained in recognizing preview image (S200). For example, the step of storing scan image (S400) may store the scan image by including, as macro information, a patient name, a patient age, a national identification number of the patient, a patient ID (or a patient number), a medical department, and a disease name obtained in recognizing preview image (S200). For example, the step of storing scan image (S400) may store the scan image by including, as macro information, a body tissue name of the corresponding sample (for example, liver tissue), whether staining is performed, a name of a staining reagent, and a specimen collection time obtained in recognizing preview image (S200). For example, the step of storing scan image (S400) may store the scan image by including, as macro information, a priority (for example, whether an urgent examination is required), a scan resolution, a scan region, and a scan method obtained in the step of recognizing preview image (S200).

In an embodiment, the step of storing scan image (S400) may store the scan image by including, as macro information, a scan resolution, a scan region, or a scan method used in the process of obtaining the scan image in the step of acquiring scan image (S300).

In an embodiment, the step of storing scan image (S400) may transmit a scan image obtained in the step of acquiring scan image (S300) to an external device based on information regarding the pathological sample obtained in the step of recognizing preview image (S200). For example, according to the information regarding the pathological sample, when the pathological sample is subject to an urgent examination, the step of storing scan image (S400) may transmit the scan image of the pathological sample to the external device in association with the information regarding the pathological sample. As a result, a medical professional who has requested the urgent examination may be provided with the image of the pathological sample with priority.

The foregoing description of the present disclosure is provided for illustrative purposes, and a person having ordinary skill in the art to which the present disclosure pertains will understand that various modifications may be easily made in other specific forms without departing from the technical spirit or essential characteristics of the present disclosure. Therefore, the embodiments described above should be understood in all respects as illustrative and not restrictive. For example, each component described as a single unit may be implemented in a distributed manner, and likewise, components described as being distributed may be implemented in a combined form.

The scope of the present disclosure should be defined by the appended claims rather than by the foregoing detailed description, and all changes or modifications derived from the meaning and scope of the claims and their equivalents should be construed as being included in the scope of the present disclosure.

1. A slide scanning apparatus comprising:
a memory;
at least one processor configured to execute at least one instruction stored in the memory; and
a door comprising a display,
wherein the at least one processor is configured to:
identify a door state comprising at least one of a closed state of the door, an open state of the door, or a fully-open state of the door, and
to control the display to output a user interface (UI) corresponding to the door state, based on the door state.

2. The apparatus of claim 1, comprising:
a slide tray slot,
wherein the slide tray is extracted from or inserted into the slide tray slot when the door is in the fully-open state.

3. The apparatus of claim 1, comprising:
a first door damper, a second door damper, a first door lock, and a second door lock,
wherein the door is fixed by the first door damper and the first door lock when the door is in the closed state, and the door is fixed by the second door damper and the second door lock when the door is in the fully-open state.

4. The apparatus of claim 1,
wherein the display includes a touch panel, and
wherein the at least one processor is configured not to acquire a user touch input to a specific region of the display when the door is in the open state or the fully-open state.

5. The apparatus of claim 1, comprising:
a slide tray slot,
wherein the user interface corresponding to the door state comprises a first GUI regarding one or more slide trays located in the slide tray slot and a second GUI regarding one or more slides located on a slide tray, and
wherein the first GUI and the second GUI comprise one or more GUIs configured to acquire a touch input when the door is in the closed state, and wherein the first GUI and the second GUI are provided so as not to acquire a touch input when the door is in the open state or the fully-open state.

6. The apparatus of claim 1, comprising:
a door lock device,
wherein the at least one processor is configured to control the door lock device to unlock the door lock device when obtaining a user input to a UI for opening the door or a UI for closing the door.

7. The apparatus of claim 1, comprising:
at least one of a forced lock-release hole or a forced fixation-release hole.

8. The apparatus of claim 7,
wherein the door is unlocked when an external force is applied through the forced lock-release hole such that a first operation bar rotates in a first direction.

9. The apparatus of claim 7,
wherein the door is unlocked when an external force is applied through the forced fixation-release hole such that a second operation bar rotates in a second direction.

10. The apparatus of claim 5,
wherein the first GUI is formed to correspond to an arrangement of a plurality of slide tray accommodation units in the slide tray slot, and the first GUI comprises a GUI indicating whether a slide tray is inserted into each of the plurality of slide tray accommodation units, and
wherein, in the first GUI, a GUI regarding a slide tray displayed through the second GUI is formed larger than GUIs regarding the other slide trays.

11. The apparatus of claim 5, wherein:
the first GUI comprises a GUI indicating at least one of:
location information of the one or more slide trays inserted into the slide tray slot;
slide tray scan progress information regarding at least a portion of the one or more slide trays;
slide tray absence information;
slot identification information;
slide tray accommodation unit identification information; or
slide tray identification information of the one or more slide trays; and
the second GUI comprises a GUI indicating at least one of:
location information of the one or more slides on a slide tray;
slide scan progress information regarding at least a portion of the one or more slides;
specimen information of the one or more slides;
code information of the one or more slides;
slide tray identification information; or
slide identification information of the one or more slides.

12. The apparatus of claim 1, comprising:
a preview camera configured to obtain a preview image of a slide tray;
a scan camera configured to obtain a slide scan image; and
a slide tray stage configured to transport a slide tray.

13. The apparatus of claim 12, wherein the at least one processor is configured to:
obtain a preview image of a slide tray;
recognize information regarding the slide tray, slides, or pathological samples from the preview image;
obtain the slide scan image based on the information regarding the slide tray, the slides, or the pathological samples; and
store the slide scan image in association with the information regarding the slide tray, the slides, or the pathological samples.

14. The apparatus of claim 12, wherein:
the slide tray stage includes a marker located at a predetermined position on the slide tray stage; and
the marker is concealed when the slide tray is accommodated in the slide try stage.
